(19) United States
(12) Patent Application Publication
(10) Pub. No.: US 2023/0397832 A1
(43) Pub. Date: Dec. 14, 2023

(54) ELECTROMAGNETIC VASCULAR FLOW SENSOR AND MEASUREMENT TECHNIQUE

(71) Applicants: Arka Das, Daytona Beach, FL (US); Eduardo Divo, Daytona Beach, FL (US); Anthony Nunez, Kissmmee, FL (US)

(72) Inventors: Arka Das, Daytona Beach, FL (US); Eduardo Divo, Daytona Beach, FL (US); Anthony Nunez, Kissmmee, FL (US)

(21) Appl. No.: 18/334,759

(22) Filed: Jun. 14, 2023

Related U.S. Application Data
(60) Provisional application No. 63/352,153, filed on Jun. 14, 2022.

(51) Int. Cl. A61B 5/0265; A61B 5/00

(52) U.S. Cl. CPC A61B 5/0265; A61B 5/6862; A61B 2562/0223

(57) ABSTRACT

A stent system can be used to measure a flow characteristic of a fluid, such as a biomagnetic fluid, flowing through a passage of a support structure forming a portion of the stent system. The stent system can include a magnetic apparatus mechanically coupled to the support structure, the magnetic apparatus configured to generate a magnetic field through the passage, the field including a component perpendicular to a direction of flow of the biological fluid. The stent system can include electrodes mechanically coupled to the support structure, where the electrodes are connected to output a potential difference generated by the flow of the biological fluid through the magnetic field indicative of a velocity of flow of the biological fluid through the passage.

### CLAIM OF PRIORITY

This patent application claims the benefit of priority of Arka Das et al., U.S. Provisional Patent Application Ser. No. 63/352,153, titled “ELECTROMAGNETIC VASCULAR FLOW SENSOR,” filed on Jun. 14, 2022 (Attorney Docket No. 4568.008PRV), which is hereby incorporated by reference herein in its entirety.

### TECHNICAL FIELD

This document pertains generally, but not by way of limitation, to stent structures, and more particularly to electromagnetic flow sensors for intravascular or extravascular stent structures, such as can be communicatively coupled or otherwise associated with another extravascular element, such as comprising excitation or measurement circuitry.

### BACKGROUND

Coronary artery disease (CAD) is the number one cause of death in the United States for both men and women. It is estimated that 13 million Americans have CAD and that 350,000 die from it each year. Metal stents can be employed along with balloon angioplasty (PTCA) to treat CAD and open narrowed coronary arteries. To reduce the risk of restenosis, the stents may be coated with therapeutic agents that are released locally. These drug-eluting stents (DES) can reduce the risk of restenosis from 20% with the use of bare metal stents to less than 5%.

### SUMMARY OF THE DISCLOSURE

Late stent thrombosis (LST), caused by a blood clot completely occluding flow through the stent, is a condition that usually requires urgent revascularization. The present inventors have recognized that a flow of a biomagnetic fluid, such as blood flow, can be measured using a stent with an electromagnetic flow sensor, such as to noninvasively detect reduced blood flow through the stent due to restenosis or thrombosis. The stent can be intravascular, such as located within a body lumen such as within an artery or vein, or the stent can be placed in an extravascular location such as encircling a structure in which a body fluid such as blood is flowing. Such a flow sensor can use Faraday's law of induction to measure blood flow. The flow sensor described herein can be used as a portion of a percutaneously delivered system (e.g., such as including a stent structure for delivery to an intravascular or extravascular location according to various examples), such as forming a portion an active implanted flow sensor, an in-situ blood analysis or separation system such as based on erythrocyte transport, or a thrombus detector, as illustrative examples.

In an example, a stent system can include a support structure defining a passage through which a biological fluid is to flow, a magnetic apparatus mechanically coupled to the support structure, the magnetic apparatus configured to generate a magnetic field through the passage, the field including a component perpendicular to a direction of flow of the biological fluid, and electrodes mechanically coupled to the support structure, the electrodes connected to output a potential difference generated by the flow of the biological fluid through the magnetic field indicative of a velocity of flow of the biological fluid through the passage. In another example, a method for measuring a flow of a biological fluid can include, within a passage defined by a stent support structure, establishing a magnetic field oriented in a first direction perpendicular to a flow direction of a biological fluid, using electrodes mechanically coupled to the stent support structure, measuring a potential across a region where the biological fluid flows, elicited by the magnetic field, and determining a velocity of the flow of the biological fluid using the potential. In the examples mentioned above, the magnetic field can be established using a permanent magnet (or multiple such magnets), an electromagnet (or multiple such magnets) or a combination of permanent magnet and electromagnetic elements.

This summary is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a diagram illustrating an example comprising a stent-based flow sensor.

FIG. 2A is a diagram illustrating an example comprising a stent-based flow sensor, the example comprising an intravascular stent structure and electrodes.

FIG. 2B is a diagram illustrating an example comprising a stent-based flow sensor, the example comprising an intravascular stent structure similar to FIG. 2A, shown at a vascular location.

FIG. 2C is a diagram illustrating an example comprising a stent-based flow sensor, the example comprising extravascular features at a vascular location.

FIG. 3A is a diagram illustrating magnetic and electromotive field directions for an example comprising a stent-based flow sensor.

FIG. 3B is a diagram showing electrical excitation and measurement interconnections that can be used in an example, the example comprising a stent-based flow sensor with a PNP type connection to receive an output frequency signal.

FIG. 4A is an exploded view of an example comprising a stent-based flow sensor showing electrodes and an electromagnet assembly.

FIG. 4B is a non-exploded view of an example comprising the stent-based flow sensor of FIG. 4A.

FIG. 4C and FIG. 4D illustrate magnetic field directions for the stent-based flow sensor of FIG. 4A.

FIG. 4E is a diagram showing electrical excitation and measurement interconnections that can be used in an example, the example comprising a stent-based flow sensor with a PNP type connection to receive an output frequency signal.

FIG. 4F is a diagram showing a signal processing topology, such as can be used to output a frequency or count indicative of a flow characteristic, such as by processing a differential electrical signal obtained using electrodes as shown in other examples herein.

FIG. 5A, FIG. 5B, FIG. 5C, and FIG. 5D show other variations comprising stent structures that can define one or more rings encircling a vascular structure, such as providing extravascular support configurations for an electromagnetic flow sensor.

FIG. 6 illustrates generally a technique, such as a measurement method for determining a flow characteristic of a biological fluid.

### DETAILED DESCRIPTION

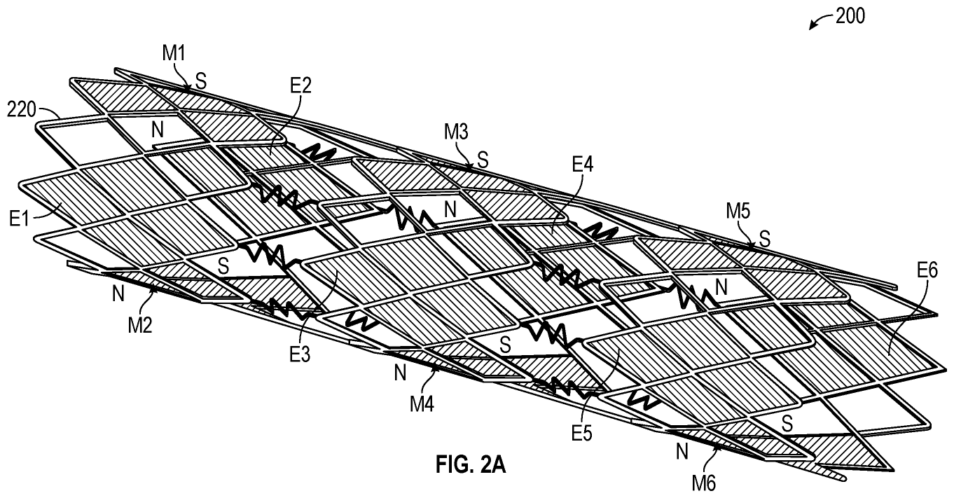

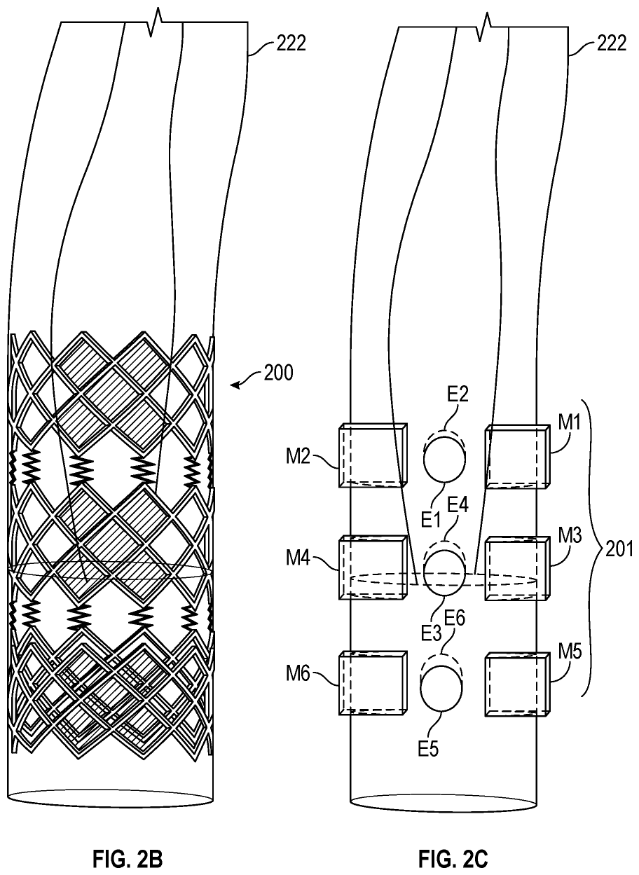

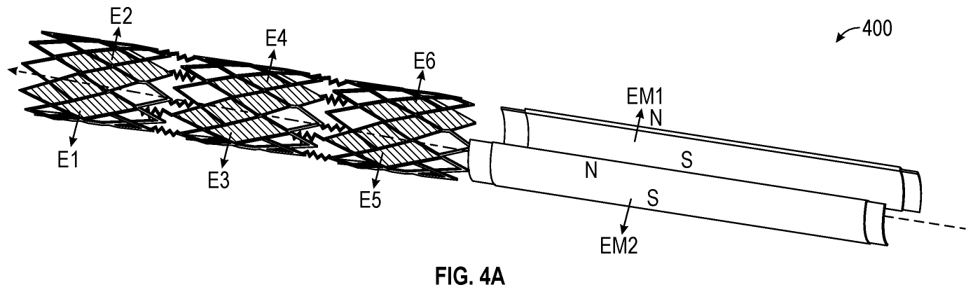

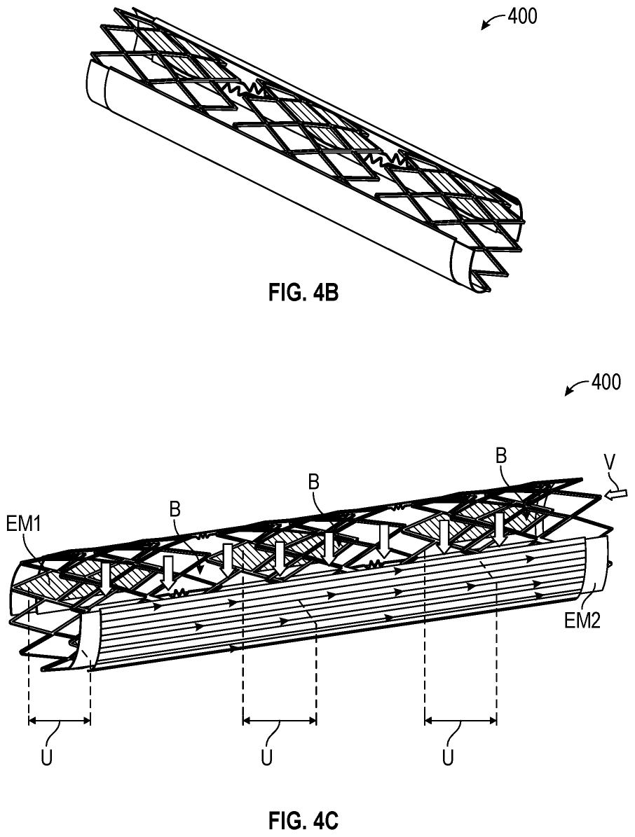

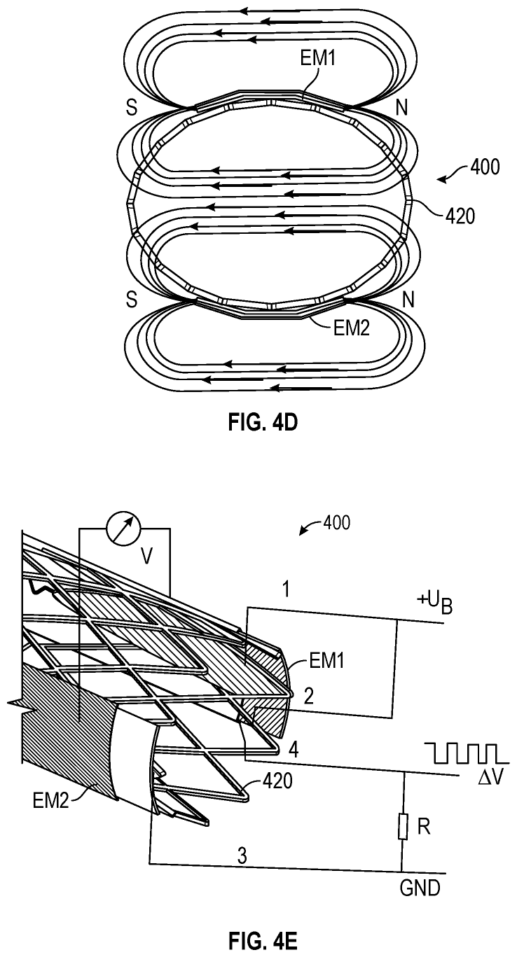

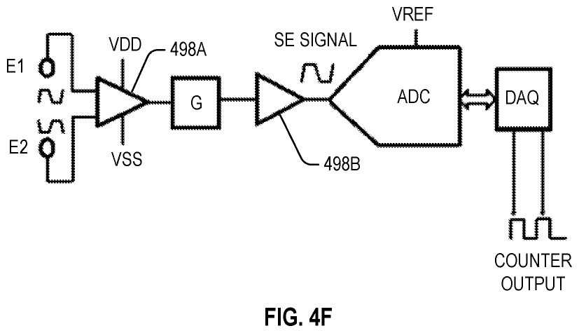

As mentioned above, a flow of a biomagnetic fluid, such as blood flow, can be measured using a stent with an electromagnetic flow sensor, such as to noninvasively detect reduced blood flow through the stent due to restenosis or thrombosis. The stent can be intravascular, such as located within a body lumen such as within an artery or vein, or the stent can be placed in an extravascular location such as encircling a structure in which a body fluid such as blood is flowing. Such a flow sensor can use Faraday's law of induction to measure blood flow. The flow sensor may be manufactured by laser cutting and Direct Metal Laser Sintering (DMLS), as examples. Two illustrative examples are described herein. Example I (e.g., FIG. 2A) uses three pairs of coupled electrodes and magnets, placed on opposite walls of the stent, such as at intersections of the struts comprising the stent structure. Example II (e.g., FIG. 4A and FIG. 4B) uses a similar electrode configuration and electromagnet coils that can be arranged to run from end to end of the extravascular device and intravascular stent.

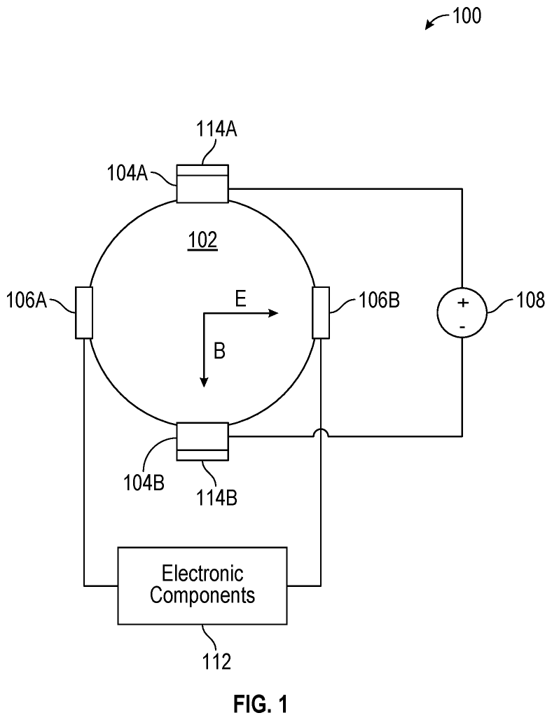

FIG. 1 is a diagram illustrating an example comprising a stent-based flow sensor 100, such as can be implemented as shown in other examples below. The flow sensor 100 can include a stent structure (e.g., expandable or collapsible scaffold) defining a stent passage 102 (e.g., a lumen), respective magnets 104A and 104B (e.g., electromagnet coils or permanent magnets), respective electrodes 106A and 106B. A voltage source 108 can be included such as to excite electromagnet coils. Optionally, magnetic core materials or respective heat sinks 114A and 114B can be provided. Measurement circuitry or other electronic components 112 can be electrically coupled with the respective electrode 106A and 106B. Some of the elements shown in FIG. 1 can be located externally, such as the electronic components 112 or voltage source 108, for example. For example, the voltage source 108 may be used to excite a coil acting as an electromagnet to generate a magnetic field (B) within the stent passage 102, or such a field can be established statically using permanent magnets for the magnets 104A and 104B. Generally, magnets 104A and 104B are located on a perimeter of the passage 102 opposite each other at first and second radial positions, respectively, such as mechanically coupled with the stent structure or formed as a portion of such structure. The electrodes 106A and 106B can also be located on the perimeter of the passage 102 opposite each other, such as offset along the perimeter at third and fourth radial positions, respectively, so that such electrodes are oriented at about ninety degrees offset from the orientation of the magnets 104A and 104B (e.g., a line between the centers of magnets 104A and 104B is ninety degrees offset from a line between the centers of electrodes 106A and 106B in plane defined by a cross section of the passage 102.

A biomagnetic fluid may be present in living organisms and its flow is influenced by the presence of a magnetic field. An example of a biomagnetic fluid is blood, which behaves as a magnetically conductive fluid, due to its complex interaction of the intercellular protein, cell membrane, and the hemoglobin, a form of iron oxide, which is present at a uniquely high concentration in the mature erythrocytes. Magnetic properties of blood are affected by factors such as a state of oxygenation. The electrodes 106A and 106B can be used measure a potential created by the blood flowing through the stent passage 102 based on a Lorentz force. The Lorentz force is the combination of electric and magnetic force acting on a point charge under an applied electromagnetic field. The force experienced by a particle of charge q moving with a velocity v under an applied electric field E and a magnetic field B is F=q(E+v×B).

Variation of this Lorentz force leads to the principle of Faraday's law of Induction. The electromagnetic flow sensor 100 works according to Faraday's law of induction. To measure a flow rate, the stent passage 102 is located in the magnetic field (B). Blood moving with velocity (V) that flows through the stent passage 102 in a direction orthogonal to the applied magnetic field induces an electromotive force in terms of potential difference (U) proportional to the mean flow velocity, which can establish a corresponding voltage across the electrodes 106A and 106B. The potential difference (U) can be monitored by electronic components 112 to identify a flow rate for the blood flowing through the stent passage 102.

The electronic components 112 may include one or more components capable of detecting or otherwise acting upon the potential difference between the electrodes 106A and 106B. For example, the electronic components 112 may include a transmitter circuit capable of conditioning and wirelessly transmitting a signal indicative of the potential difference between the electrodes 106A and 106B such that one or more remote systems may receive data indicative of the value to identify a flow rate of the blood through the stent passage 102. In another example, the electronic components 112 may include an analog circuit, digital circuit, or mixed signal circuitry comprising analog and digital circuits. As examples, the electronic components 112 can include data storage circuits or devices, switches, relays, a controller, a digital signal processor (DSP), an application specific integrated circuit (ASIC), or any other components. For example, the electronic components 112 may include an ASIC capable of interpreting the potential difference between the electrodes 106A and 106B to detect a decrease in blood flow, in response to which an indicator may be transmitted or otherwise output to provide an indication of a decrease in blood flow to one or more remote users or systems.

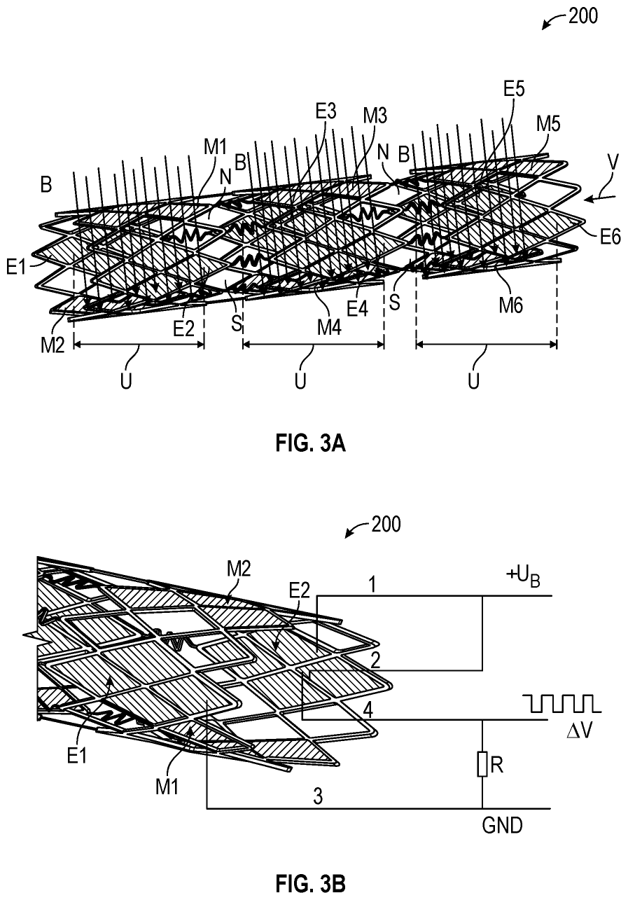

FIG. 2A is a diagram illustrating an example comprising a stent-based flow sensor 200, the example comprising an intravascular stent structure and electrodes. FIG. 2B is a diagram illustrating an example comprising a stent-based flow sensor 200, the example comprising an intravascular stent structure similar to FIG. 2A, shown at a vascular location within a vessel 222. Generally, in the examples shown in FIG. 2A, FIG. 0.2B, FIG. 3A, and FIG. 3B, the stent-based flow sensor 200 may be configured in such a way that a noble element (e.g., gold or silver) sputtered pair of electrodes (e.g., E1, E2; E3, E4; E5, E6) are placed along opposite walls and permanent magnets (e.g., M1, M2; M3, M4; M5, M6) are oriented to establish fields having opposite polarities are placed on opposite surfaces of the stent. A pair of electrodes and corresponding permanent magnets are located such that a potential established across the electrodes is established in a direction orthogonal to the magnetic field orientation, such as by locating the electrodes in an orientation around the stent that is oriented ninety degrees apart from an axis between the permanent magnets, either on the inside lumen of the stent or at the outside surface of the stent. Each assembly of such a pair of electrodes (e.g., E1 and E2) and permanent magnets (e.g., M1 and M2) may be referred to as a “cluster.” Each pair of electrodes and permanent magnets can be placed at the opposite walls of the stent and permanent magnets are showing opposite polarities to each other (for example, N-S field of attraction among them by creating a perpendicular magnetic field passing through the stent geometry).

The examples of FIG. 2A and FIG. 2B comprise a stent support structure 220 for an intravascular location, but a similar configuration could be used at an extravascular location, such as illustrated in FIG. 2C. For example, FIG. 2C is a diagram illustrating an example comprising a stent-based flow sensor, the example comprising extravascular features at a vascular location. In FIG. 2C, permanent magnets M1, M2, M3, M4, M5, and M6 can form portions of clusters similarly to FIG. 2A and FIG. 2B. Corresponding electrodes E1, E2, E3, E4, E5, and E6 can be used for measuring potentials induced by a flow of a biomagnetic fluid through a lumen of a vessel 222.

FIG. 3A is a diagram illustrating magnetic and electromotive field directions for an example comprising a stent-based flow sensor. As mentioned above, stent-based flow sensor 200 may be patterned with three such repeated sets of clusters in series:
Cluster 1: (E1, E2): Pair of electrodes and (M1, M2) pair of permanent magnets
Cluster 2: (E3, E4): Pair of electrodes and (M3, M4) pair of permanent magnets
Cluster 3: (E5, E6): Pair of electrodes and (M5, M6) pair of permanent magnets

When a conductive fluid flows through the stent with a velocity V, with a magnetic field B acting perpendicular to the flow from top to bottom (as oriented in the illustrative example of FIG. 3A), then a potential difference U is created across the pair of electrodes placed in the fluid at the respective stage. This potential difference can be correlated with the velocity of the fluid field flowing through the stent as shown in FIG. 3A. In this example, an error associated with the measurements of the induced potential difference U at each pair of electrodes in each cluster can be reduced or even minimized by determining a mean value or other central tendency of the induced potential differences established at the three clusters, such as for each sample. The electrodes of different clusters placed at a same wall of the stent may be connected in series through a conductive wire sputtered with a same noble element as the electrodes, for example.

FIG. 3B is a diagram showing electrical excitation and measurement interconnections that can be used in an example, the example comprising a stent-based flow sensor 200 with a PNP type connection to receive an output frequency signal.

For examples having conductive interconnections (e.g., wires or leads), the electrode cluster assembly may be connected in a manner establishing a negative-positive-negative (NPN) configuration or a positive-negative-positive (PNP) configuration, using a pull-up element (e.g., a resistor) to output a square wave signal with varying frequency proportional to the flow rate of the conductive fluid flowing through the tube. As an illustration, nodes 1 and 3 can be connected to respective electrodes in an electrode pay (e.g., E1 and E2). Nodes 2 and 4 can be connected to each other using a switch structure (e.g., a solid-state switch such as using a transistor network). For example, such a switch can be energized by an excitation source (e.g., an external AC source or an AC source included as a portion of implantable circuit), such as toggling between open and closed states. Such toggling can provide a time-varying voltage representative of the flow characteristic, +U_{B}. The time-varying voltage representative of the flow characteristic can be processed, such as using a signal processing topology (e.g., “signal chain”) as shown illustratively in the example of FIG. 4B below.

Aspects of the example (e.g., “Example I”) of FIG. 2A, FIG. 2B, FIG. 3A, and FIG. 3B, can include: no electric current is required for charging the magnets (e.g., these examples use permanent magnetic structures); no inductance circuit is necessary for wirelessly powering permanent magnets; no heating effect is produced by the magnet by joule heating; no heat sink is required for the permanent magnets; no electrical insulation and shielding need be included between the permanent magnets and the electrode assembly; and no core material selection is needed for the permanent magnets.

FIG. 4A is an exploded view of an example comprising a stent-based flow sensor 400 showing electrodes E1, E2, E3, E4, E5, and E6, and an electromagnet assembly comprises electromagnet coils EM1 and EM2. FIG. 4B is a non-exploded view of an example comprising the stent-based flow sensor 400 of FIG. 4A, with the electromagnet assembly located concentrically with the electrodes. FIG. 4C and FIG. 4D illustrate magnetic field directions for the stent-based flow sensor 400 of FIG. 4A, with FIG. 4D showing cross sectional views of field orientation around electromagnet coils EM1 and EM2, located opposite each other on or within a stent structure 420.

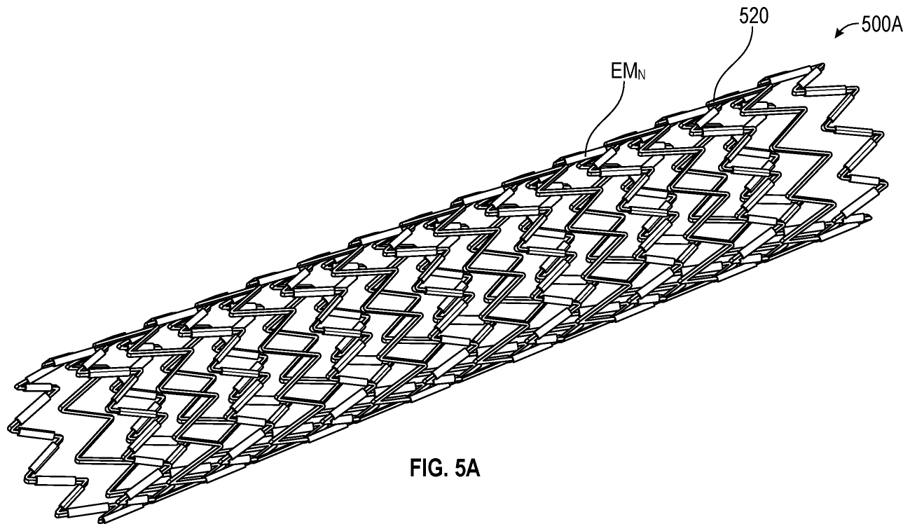

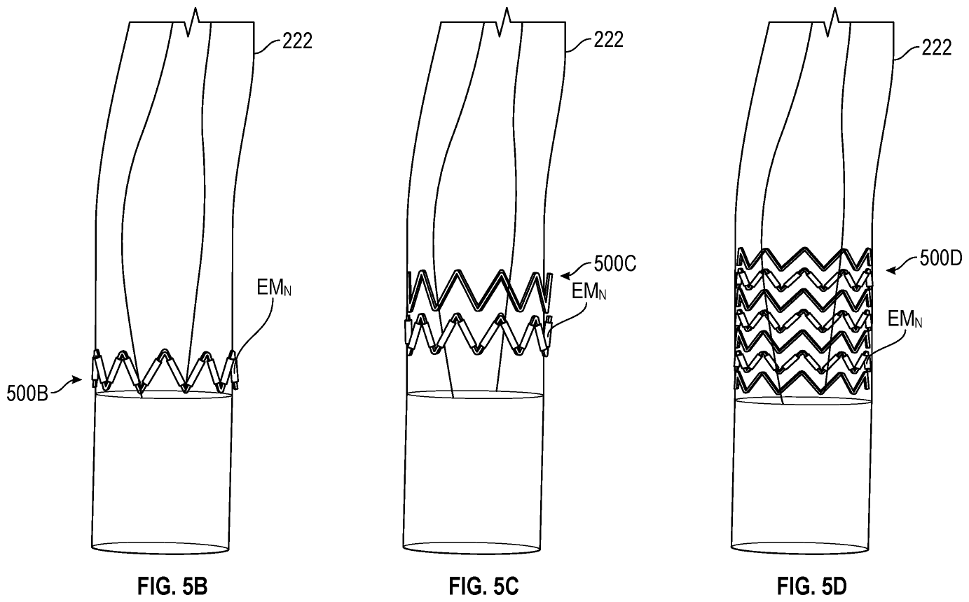

In this example, rather than the permanent magnet clusters of Example I (e.g., FIG. 2A, FIG. 2B, FIG. 3A, FIG. 3B), two electromagnets are used, such as arranged running from end to end of the stent structure. These electromagnets are placed on the walls of the stent in such a way that generated magnetic field is orthogonal to the flow direction of the conductive fluid as shown in FIG. 4C. These electromagnets may be wound with coils either in the horizontal or vertical direction along the walls of the device and the struts of the stent, producing a N-S field orthogonal to the conductive fluid flowing along the length of the stent as shown illustratively in the example of FIG. 5A, FIG. 5B, FIG. 5C and FIG. 5D. As an illustration, three pairs of electrode assemblies may be included between these electromagnets and the stent wall. The Example II flow sensor can otherwise function or be operated in a similar manner to the Example I flow sensor described above.

FIG. 4E is a diagram showing electrical excitation and measurement interconnections that can be used in an example, the example comprising a stent-based flow sensor with a PNP type connection to receive an output frequency signal. A variable voltage magnitude, such as corresponding to an AC excitation waveform, can be applied to electromagnet coils EM1 or EM2 (or both). Generally, in FIG. 4E, measurement and excitation nodes can be connected in a manner similar to FIG. 3B. For example, nodes 1 and 3 can be connected to respective electrodes, and nodes 2 and 4 can be connected to each other such as in response to applied excitation using a solid-state switch. An amplitude or frequency (or both) of the excitation signal can be similar to that used in relation to the example of FIG. 3B. However, such excitation can also be used to energize the electromagnet coils EM1 and EM2 contemporaneously. In this manner, an electromagnetic flux density established inside the sensor can be modulated by varying such amplitude or frequency. The stent structure need not serve as a conductor.

FIG. 4F is a diagram showing a signal processing topology, such as can be used to output a frequency or count (“COUNTER OUTPUT”) indicative of a flow characteristic, such as by processing a differential electrical signal obtained using electrodes (e.g., “E1” and “E2”) as shown in other examples herein. Generally, a differential signal can be established across nodes E1 and E2, such as corresponding to a chopped or time-varying representation of an induced voltage +U_{B}, as mentioned elsewhere herein. The induced voltage +U B may be quite weak, such as on the order of 70 microvolts to 3 or 4 millivolts, as an illustration. Accordingly, respective amplifier stages can be used such as amplifiers 498A and 498B, and an instrumentation amplifier (e.g., having a gain, “G,” from a range of 10× to 100×, as an illustration), before a representation of the induced voltage is provided to an analog to digital converter (“ADC”). The ADC can receive a single-ended representation of the induced voltage from the second amplifier 498B and a digital representation of the induced voltage can be provided to a data acquisition system. The ADC can include or can be referenced to a precision voltage reference VREF (e.g., an output of a bandgap or other precision reference), such as separate from a positive supply node VDD or a negative supply node VSS. A time-series representation of ADC output conversions can be transformed into a frequency or digital counter output at the DAQ (e.g., a digital pulse train corresponding to a measured characteristic such as flow rate.

Aspects of the configuration of Example II (e.g., FIG. 4A, FIG. 4B, FIG. 4C, FIG. 4D, FIG. 4E) can include: adjustable and uniform magnetic field; for a fluid moving with a velocity V, can vary the magnetic field and a corresponding magnitude of AU generated at each electrode can be modulated without requiring an external amplification or gain at the processing circuit; an electromagnet configuration or excitation can be tailored in shape; and may be lower in cost to manufacture as compared to Example I (e.g., FIG. 2A, FIG. 2B, FIG. 3A, FIG. 3B), depending on materials and configuration. Generally, the stent-based flow sensor structures discussed in Examples I and II can be used for a variety of applications. For example, such structures can be used to detect intimal hyperplasia and a restenosis condition. The clusters in the stent (Example I) and electromagnets in the stent (Example II) will detect a change in the electromotive force or induced EMF on each electrode in response to cellular build-up in the lumen. Referring back to FIG. 1, to measure a flow rate, restenosis occurring in the passage 102 can be located in the magnetic field (B). Blood moving with velocity (V′) that flows through the partially obstructed passage 102 in a direction orthogonal to the applied magnetic field induces an electromotive force in terms of a potential difference (U′) proportional to the mean flow velocity, which establishes a corresponding voltage across the electrodes 106A and 106B. The relative change in potential difference ∥U-U′∥ induced on each electrode for each heart cycle can be acquired to determine a presence or degree of intimal hyperplasia, as an illustrative example. Generally, the stent-based flow sensor structures are applicable to various locations, such as in relation to cerebrovascular stents, coronary stents, peripheral stents, venous stents, carotid stents, or aortic stents, as illustrative but non-restrictive examples.

FIG. 5A, FIG. 5B, FIG. 5C, and FIG. 5D show other variations comprising stent structures that can define one or more rings encircling a vascular or other body structure defining a lumen through which a biomagnetic fluid can flow. Generally, the examples of FIG. 5A, FIG. 5B, FIG. 5C, and FIG. 5D show structures that can provide extravascular support configurations for an electromagnetic flow sensor. For example, in FIG. 5A, an assembly 500A can include a stent structure 520 having “rib” structures comprising respective magnet or electromagnetic elements (e.g., EM N). Such elements can be excited in a controlled manner, such as according to a specified sequence of regions being energized (and resulting electromagnetic flux being established within a passage of the stent structure 520). An extravascular configuration can include a single ring assembly 500B as shown in FIG. 5B, with one or more electromagnet coils (e.g., EM N) as rib structures included on respective segments. In an example, as shown in FIG. 5C, multiple rings can be included as a portion of a system 500C, such as comprising a ring without electromagnetic elements, and a ring with one or more electromagnet coils (e.g., EM N) as rib structures included on respective segments. Such an approach can be modular, allowing different combinations of ring configurations. For example, in FIG. 5D, a system 500D can include several rings, such as alternating between rings having electromagnet structures such as an electromagnet coil EM N and rings not having or not using such structures. For example, alternating between rings having electromagnetic elements and lacking such elements (or otherwise staggering the locations of such elements) can help suppress or inhibit mutual inductive effects where individual coil performance or parameters are affected by such mutual inductive effects.

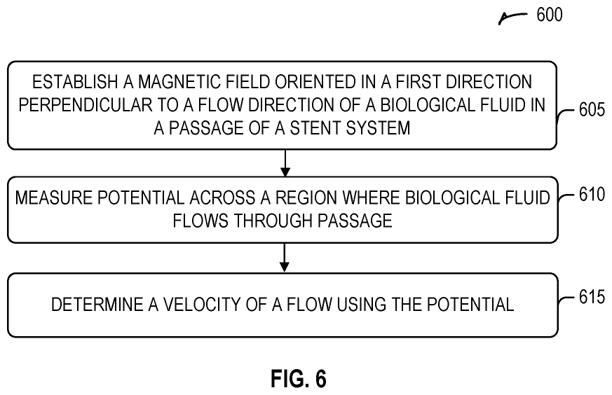

FIG. 6 illustrates generally a technique, such as a measurement method 600 for determining a flow characteristic of a biological fluid. At 605, the measurement method 600 can include establishing a magnetic field oriented in a first direction perpendicular to a flow direction of a biological fluid, such as establishing the magnetic field within a passage defined by a stent support structure as shown and described elsewhere herein. At 610, a potential can be measured, the potential developed across a region where the biologic fluid flows through the passage, and the potential elicited by the magnetic field. At 615, a velocity of the flow can be determined using the potential. Such a determination can be made using circuitry forming a portion of an implantable apparatus, or using an external device communicatively coupled with an implantable stent system.

### Various Notes

Each of the non-limiting aspects in this document can stand on its own or can be combined in various permutations or combinations with one or more of the other aspects or other subject matter described in this document.

The above detailed description includes references to the accompanying drawings, which form a part of the detailed description. The drawings show, by way of illustration, specific embodiments in which the invention can be practiced. These embodiments are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc., are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Such instructions can be read and executed by one or more processors to enable performance of operations comprising a method, for example. The instructions are in any suitable form, such as but not limited to source code, compiled code, interpreted code, executable code, static code, dynamic code, and the like. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A stent system, comprising:
a support structure defining a passage through which a biological fluid is to flow;
a magnetic apparatus mechanically coupled to the support structure, the magnetic apparatus configured to generate a magnetic field through the passage, the field including a component perpendicular to a direction of flow of the biological fluid; and
electrodes mechanically coupled to the support structure, the electrodes connected to output a potential difference generated by the flow of the biological fluid through the magnetic field indicative of a velocity of flow of the biological fluid through the passage.

2. The stent system of claim 1, wherein the magnetic apparatus comprises a permanent magnet structure.

3. The stent system of claim 2, wherein the permanent magnet structure comprise a first permanent magnet positioned at a first radial position on a perimeter of the passage, and a second permanent magnet positioned at a second radial position on the perimeter of the passage opposite the first position.

4. The stent system of claim 3, wherein the electrodes comprise a first electrode positioned at a third radial position on the perimeter of the passage between the first and the second permanent magnets, and a second electrode positioned at a fourth radial position on the perimeter of the passage opposite the third position.

5. The stent system of claim 1, wherein the magnetic apparatus comprises an electromagnet structure comprising one or more coils excited to generate the magnetic field.

6. The stent system of claim 5, wherein the electromagnet structure comprises a first electromagnet positioned at a first radial position on a perimeter of the passage, and a second electromagnet positioned at a second radial position on the perimeter of the passage opposite the first position.

7. The stent system of claim 6, wherein the electrodes comprise a first electrode positioned at a third radial position on the perimeter of the passage between the first and the second electromagnets, and a second electrode positioned at a fourth radial position on the perimeter of the passage opposite the third position.

8. The stent system of claim 1, wherein the support structure is configured for delivery to an extravascular location.

9. The stent system of claim 8, wherein the support structure is at least one of expandable or collapsible.

10. The stent system of claim 1, wherein the support structure is configured for delivery to an intravascular location.

11. The stent system of claim 10, wherein the support structure is expandable after delivery to an intravascular location.

12. A method for measuring a flow of a biological fluid, the method comprising:
within a passage defined by a stent support structure, establishing a magnetic field oriented in a first direction perpendicular to a flow direction of a biological fluid;
using electrodes mechanically coupled to the stent support structure, measuring a potential across a region where the biological fluid flows, elicited by the magnetic field; and
determining a velocity of the flow of the biological fluid using the potential.

13. The method of claim 12, wherein the electrodes are located at respective radial positions along the stent support structure that are offset by ninety degrees from corresponding radial positions of magnets used to establish the magnetic field.

14. The method of claim 13, wherein the magnets comprise permanent magnets.

15. The method of claim 13, wherein the magnets comprise electromagnet structures.

16. The method of claim 15, wherein establishing the magnetic field comprises exciting the electromagnet structures using an alternating current waveform.

17. The method of claim 16, wherein establishing the magnetic field comprises selecting at least one of an amplitude or a frequency of an excitation waveform to elicit a measurable potential for the electrodes.

18. The method of claim 13, comprising deploying the stent support structure at an extravascular location.

19. The method of claim 13, comprising deploying the stent support structure at an intravascular location.

20. A method for measuring a flow of a biological fluid, the method comprising:
within a passage defined by a stent support structure, a means for establishing a magnetic field oriented in a first direction perpendicular to a flow direction of a biological fluid;
a means for measuring a potential across a region where the biological fluid flows, elicited by the magnetic field; and
a means for determining a velocity of the flow of the biological fluid using the potential.
